(19) United States
(12) Patent Application Publication
(10) Pub. No.: US 2008/0089890 A1
(43) Pub. Date: Apr. 17, 2008

(54) METHODS OF POLYPEPTIDE PRODUCTION

(75) Inventors: Elena Brin, San Diego, CA (US); Daniel D. Giroux, Cardiff by the Sea, CA (US); Drake M. LaFace, San Diego, CA (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(73) Assignee: Canji, Inc.

(21) Appl. No.: 11/864,608

(22) Filed: Sep. 28, 2007

Related U.S. Application Data
(60) Provisional application No. 60/848,551, filed on Sep. 29, 2006.

(51) Int. Cl. C12P 21/00; A61K 38/02; A61K 39/395

(52) U.S. Cl. 424/141.1; 435/69.1; 435/69.6; 514/2

(57) ABSTRACT

The present invention relates to methods and compositions for adenovirus mediated production of a polypeptide of interest in replication non-permissive cells. The invention also relates to compositions resulting from the methods and uses according to the invention.

### REFERENCE TO CROSS RELATED APPLICATIONS

This application claims the benefit of priority under 35 USC 119(e) of provisional patent application U.S. Ser. No.: 60/848,551 filed Sep. 29, 2006, the disclosure of which is hereby incorporated by reference in its entirety.

### FIELD OF THE INVENTION

The present invention relates to methods and compositions for adenovirus mediated production of a polypeptide.

### BACKGROUND OF THE INVENTION

The expression of human recombinant polypeptides in various cells is well known. Production systems for recombinant polypeptides include those based on the use of bacteria, yeasts, fungi, insect cells, plant cells and mammalian cells. However, despite these advances, some production systems are not optimal, or are only suited for production of specific classes of polypeptides. For example, polypeptides that require post-translational modifications such as glycosylation, g-carboxylation, or g-hydroxylation cannot be produced in prokaryotic production systems. Another problem associated with the use of prokaryotic expression systems is the incorrect folding of the polypeptide to be produced leading to insoluble inclusion bodies in some cases. Moreover, glycosylation in eukaryotic insect cells is different than in mammalian cells, resulting in the production of altered glycoproteins. Therefore, eukaryotic systems are an improvement in the production of, in particular, eukaryote derived polypeptides.

Recombinant adenoviruses have been used for functional polypeptide expression in mammalian cells including those of human and nonhuman origin. Recombinant adenovirus vectors have desirable features for gene delivery, including wide tissue and cell tropism, the capacity to accommodate large expression cassettes and high transduction efficiency.

For pharmaceutical development and commercial manufacture of viral vectors, the vector-cell line combination also must be amenable to scale-up and provide material of sufficient quality and purity. High-level polypeptide production by recombinant adenovirus vectors is expected in their replication permissive host cells, such as the human embryonic kidney 293 cell (HEK293) cell line. This is attributed primarily to the permissiveness of HEK293 cells to human recombinant adenovirus infection and their ability to support viral DNA replication by providing missing adenoviral proteins. However, the HEK293 cells tend to suffer from cytopathic effect (CPE) as a result of virus replication. Under such conditions, the host cell function is compromised and the culture viability will be reduced. Consequently, newly synthesized polypeptides may not be efficiently expressed in large quantities and may not be processed properly at the post-translational level. Therefore, the usefulness of HEK293 cells for the expression of complex targets such as secreted polypeptides could be limited.

Other commonly used human cell lines include A549 and HeLa cells. The A549 cell line, though readily infected by human adenoviruses, cannot tolerate high doses of viral infection. It is also difficult to propagate A549 cells in serum free suspension culture, one of the preferred modes for scaleable polypeptide production. Although HeLa cells are permissive to adenovirus infection and replication and grow rapidly in suspension culture, they normally require higher levels of virus to achieve efficient vector transfer.

Accordingly, there is a need for more efficient cell lines for recombinant adenovirus mediated polypeptide expression in mammalian cells.

### 1. SUMMARY OF THE INVENTION

The present invention provides methods and compositions for recombinant adenovirus mediated expression of a polypeptide in a mammalian cell. Specifically, the invention provides methods and compositions for recombinant adenovirus mediated expression of a polypeptide in a replication non-permissive mammalian cell.

The invention provides a method for production of a polypetide, said method comprising culturing a replication non-permissive mammalian cell containing a recombinant adenovirus comprising a nucleotide sequence encoding a polypeptide of interest under conditions so as to permit expression of the polypeptide in said cell.

The invention provides a method for production of a polypeptide, said method comprising culturing a replication non-permissive mammalian cell containing a recombinant adenovirus comprising a nucleotide sequence encoding a polypeptide of interest under conditions so as to permit expression of the polypeptide in said cell and, optionally, harvesting said cell and/or recovering said polypeptide of interest.

In some embodiments of the invention, the replication non-permissive mammalian cell is a hepatocellular cell, such as, for example, a HepG2 cell (ATCC No.: HB8065).

In a specific embodiment of the invention, the polypeptide of interest may be produced by culturing hepatocellular carcinoma cells; optionally adding fresh growth medium to the cells; inoculating the cells with the recombinant adenovirus; incubating the inoculated cells for a time sufficient to allow for the expression of the polypeptide of interest; optionally adding fresh growth medium to the inoculated cells; and optionally harvesting the polypeptide of interest from the infected cells and/or the medium.

In some embodiments of the invention, the polypeptide of interest is a cytokine or antibody.

The present invention also provides a hepatocellular carcinoma cell infected with a recombinant adenovirus according to the invention, as well as a culture of such cells comprising said infected cells and a culture medium. The polypeptide of interest can be optionally isolated from such a culture of cells, e.g., from the cells, from the culture medium or from both.

The invention also provides compositions resulting from the methods and uses according to the invention, especially when combined in a pharmaceutical composition including suitable excipients and/ or adjuvants.

### 2. DETAILED DESCRIPTION OF THE INVENTION

The present invention provides a method for production of a polypeptide of interest in a mammalian cell, said method comprising culturing a replication non-permissive mammalian cell containing a recombinant adenovirus comprising a nucleotide sequence encoding a polypeptide of interest under conditions so as to permit expression of the polypeptide in said cell. The invention also provides compositions resulting from the methods and uses of the invention.

The present inventions provides a simple, rapid and/or efficient method for production of a polypeptide of interest in a replication non-permissive mammalian cell containing a recombinant adenovirus comprising a nucleotide sequence encoding the polypeptide of interest. Generally, the present invention provides one or more of the following advantages: larger polypeptide yield, use of continuous cultures (e.g., about twenty or about thirty days or more), postranslational modification during polypeptide production, ability to produce the polypeptide of interest in a serum free media allowing for simplification of purification, quick adoption to production of the polypeptide of interest and/or use in production of non-homogenous multimers (e.g., antibodies, IL-23). By way of example, and not limitation, production of polypeptides by the present invention can result in about a five fold increase in yield per ml. In addition, as the present invention provides the capacity to harvest proteins (e.g., from supernantants) over time, production of polypeptides by the present invention can result in about a 10-fold increase in total protein production.

In one embodiment the cell line is a human hepatocellular cell line (e.g., HepG2) and the replication defective adenovirus comprising a heterologous sequence is derived from a human adenovirus serotype 5. By way of example and not limitation, the cells may be grown to a high density in a bioreactor, infected and continuously perfused with fresh media and the polypeptide encoded by the heterologous sequence is continuously harvested from the spent media for about twenty or thirty days.

Conventional molecular biology, microbiology, and recombinant DNA techniques that may be employed are within the skill of the art. Such techniques are explained in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook, et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985)); Transcription And Translation (B. D. Hames & S. J. Higgins, eds. (1984)); Animal Cell Culture (R. I. Freshney, ed. (1986)); Immobilized Cells And Enzymes (IRL Press, (1986)); B. Perbal, A Practical Guide To Molecular Cloning (1984); F. M. Ausubel, et al. (eds.), Current Protocols in Molecular Biology, John Wiley & Sons, Inc. (1994).

Recombinant Adenovirus

The recombinant adenoviruses that may be used in the practice of the invention comprise adenoviral nucleotide sequences and optionally, one or more heterologous nucleotide sequences encoding a polypeptide of interest. As used herein, the term “recombinant adenovirus” refers to viruses of the genus adenoviridiae, capable of infecting a cell, whose viral genomes have been modified through conventional recombinant DNA techniques. The term recombinant adenovirus also includes chimeric (or even multimeric) vectors, i.e. vectors constructed using complementary coding sequences from more than one viral subtype. In a preferred embodiment, the recombinant adenovirus is a replication-defective adenovirus. In one embodiment the recombinant adenovirus vector is derived from a human adenovirus serotype 5 and comprises deletions of the E1a, E1b and protein IX functions, the E3 region and optionally the E2b region. Examples of replication defective adenovirus may be found in U.S. Pat. Nos.: 6,210,939, 5,932,210, herein incorporated by reference in their entirety.

In accordance with the invention, the recombinant adenovirus comprises an adenoviral genome or a portion thereof obtained and/or derived from any adenoviridae or a combination of adenoviridae. As used herein, the term “adenoviridae” refers collectively to animal adenoviruses of the genus mastadenovirus including but not limited to human, bovine, ovine, equine, canine, porcine, murine and simian adenovirus subgenera. In particular, human adenoviruses includes the A-F subgenera as well as the individual serotypes thereof, including but not limited to human adenovirus types 1, 2, 3, 4, 4a, 5, 6, 7, 7a, 7d, 8, 9, 10, 11 (Ad11A and Ad11P), 12, 13, 14, 15, 16, 17, 18, 19, 20, 21, 22, 23, 24, 25, 26, 27, 28, 29, 30, 31, 32, 33, 34, 34a, 35, 35p, 36, 37, 38, 39, 40, 41, 42, 43, 44, 45, 46, 47, 48, and 91.

The recombinant adenoviruses to be used according to the invention comprise an “expression cassette” comprising a nucleotide sequence capable of directing the transcription and translation of a heterologous coding sequence and the heterologous coding sequence to be expressed. An expression cassette comprises a regulatory element operably linked to a heterologous coding sequence so as to achieve expression of the polypeptide product of interest encoded by said heterologous coding sequence in the cell.

As used herein, the term “heterologous” in the context of nucleic acid sequences, amino acid sequences and antigens refers to nucleic acid sequences, amino acid sequences and antigens that are foreign and are not naturally found associated with a particular adenovirus.

As used herein, the term “operably linked” refers to a linkage of nucleotide sequence elements in a functional relationship. A nucleic acid sequence is “operably linked” when it is placed into a functional relationship with another nucleic acid sequence. For instance, a promoter or enhancer is operably linked to a coding sequence if it affects the transcription of the coding sequence. Operably linked means that the nucleotide sequences being linked are typically contiguous. However, as enhancers generally function when separated from the promoter by several kilobases and intronic sequences may be of variable lengths, some polynucleotide elements may be operably linked but not directly flanked and may even function in trans from a different allele or chromosome.

As used herein, the term “regulatory element” refers to promoters, enhancers, transcription terminators, insulator regions, silencing region, polyadenylation sites, and the like. The term “promoter” is used in its conventional sense to refer to a nucleotide sequence at which the initiation and rate of transcription of a coding sequence is controlled. The promoter contains the site at which RNA polymerase binds and also contains sites for the binding of regulatory factors (such as repressors or transcription factors). Promoters may be naturally occurring or synthetic. When the vector to be employed is a viral vector, the promoters may be endogenous to the virus or derived from other sources. The regulatory elements may be arranged so as to allow, enhance or facilitate expression of the transgene only in a particular cell type. For example, the expression cassette may be designed so that the transgene is under control of a promoter which is constitutively active, or temporally controlled (temporal promoters), activated in response to external stimuli (inducible), active in particular cell type or cell state (selective) constitutive promoters, temporal viral promoters or regulatable promoters. By way of example, and not limitation, an E1a defective human rAd5 vector may be used. (see, e.g., U.S. Pat. No. 6,989,268, herein incorporated by reference in its entirety).

The recombinant adenoviruses that may be used in the practice of the invention may be propagated by methods known in the art. In a preferred embodiment the recombinant adenoviruses is a replication-defective adenovirus. By way of example, but not limitation, the replication-defective adenovirus may be propagated in 293 cells. Infected cell lysate may be used as the inoculum in the present invention or the replication-defective adenovirus may be purified by methods known in the art. By way of example, the recombinant adenovirus may be purified by column chromatography in substantial accordance with the process of (Huyghe et al., 1995b) as described in Shabram, et al., U.S. Pat. No. 5,837,520 issued Nov. 17, 1998; see also U.S. Pat. No. 6,2661,823, the disclosures of which are herein incorporated by reference.

Polypetides

A recombinant gene product according to the present invention is the polypeptide that is sought to be expressed and harvested in high amount. It may be any polypeptide of interest, e.g. therapeutic polypeptides such as interleukins or enzymes or subunits of multimeric polypeptides such as antibodies or fragments thereof. The recombinant product gene may include all or part of a signal coding sequence portion allowing secretion of the once expressed polypeptide from the host producer cell. The recombinant adenoviruses to be utilized in the present invention may incorporate any heterologous nucleotide sequence, including genes or portions of genes.

“Polypeptide” refers to polymers of amino acids of any length. The polypeptide (or protein, or proteinaceous molecule, the terms are used interchangeably herein) of interest can be any polypeptide. Generally, a polypeptide with desired therapeutic and/or prophylactic and/or diagnostic purposes may be a preferred polypeptide of interest. In accordance with these embodiments, the heterologous nucleotide sequence may encode a moiety, peptide, polypeptide or protein possessing a desired biological property or activity.

In an embodiment of the invention, such a heterologous nucleotide sequence may encode a biological response modifier including, for example, a cytokine, cytokine receptor, hormone, growth factor or growth factor receptor. Non-limiting examples of such biological response modifiers include interferon (IFN)-alpha (e.g., IFN-alpha2b), IFN-beta, IFN gamma, interleukin (IL-1), IL-2, IL-3, IL-4, IL-5, IL-6, IL-7, IL-9, IL-10, IL-12, IL-15, IL-18, IL-23, erythropoietin (EPO), basic fibroblast growth factor (bFGF), acidic fibroblast growth factor (aFGF), vascular endothelial growth factor (VEGF), platelet derived growth factor (PDGF), epidermal growth factor (EGF), thymic stromal lymphopoietin (TSLP), TNFR and TNFR ligand superfamily members including TNFRSF 18 and TNFSFI8. In other embodiments, the heterologous nucleotide sequence encodes an antibody. In yet other embodiments, the heterologous nucleotide sequence encodes a chimeric or fusion protein. In certain embodiments, the heterologous nucleotide sequence encodes an antigenic polypeptide, a polypeptide or peptide that may be used to immunize a subject. In a preferred embodiment the polypeptide is a soluble polypeptide.

The desirable size of the inserted non-adenovirus or heterologous nucleotide sequence encoding the polypeptide of interest is limited to that which permits packaging of the recombinant adenovirus vector into virions, and depends on the size of retained adenovirus sequences. The genome of a human adenovirus is approximately 36 kilobase pairs in length (measured to be 35938 nucleotides in length by Davison et al. (2003) J. Gen. Virology 84 (Pt 11), 2895-2908). The total size of the recombinant adenovirus to be packaged into virions should be about 37735 nucleotides in length (about 105% of the normal genome length).

Cells

The cells utilized in the present invention may be any replication non-permissive mammalian cell. A replication non-permissive mammalian cell refers to a cell that does not permit adenoviral replication. In a preferred embodiment, the replication non-permissive mammalian cell does not permit the replication of replication defective adenovirus. Such cells may be naturally occurring or modified to become replication non-permissive. Examples of such cells include but are not limited to, HepG2 cells (ATCC number HB-8065), Hep3B cells (ATCC number HB-8064) and 4T1 murine mammary carcinoma cell line (ATCC number CRL-2539). In a preferred embodiment the cells are HepG2. By way of example, and not limitation, the cells can be HepG2 cells and the replication defective adenovirus is dervived from a human adenovirus serotype 5.

In a particularly preferred embodiment, hepatocellular carcinoma cells are used for production of a polypeptide of interest. Hepatocellular carcinoma cells according to the invention may be any permanent cell line derived from a hepatocellular liver carcinoma tissue sample. A permanent hepatocellular carcinoma cell line can be derived from a single clone obtained from a liver carcinoma sample. Permanent cells, according to the present invention, are those cells that have been genetically modified in some way so that the cell is able to continue growing permanently in cell culture. In this context, “growing permanently” in culture means that such a cell is capable of growing in culture for many generations (cell doublings), for instance at least 50 generations, preferably at least 100 generations, more preferably for still more generations, most preferably such a cell can be cultured for an indefinite period.

In accordance with the present invention, a hepatocellular carcinoma cell is used for efficient production of the polypeptide of interest. In a preferred embodiment of the invention, the hepatocellular liver carcinoma cell is a human cell. In a specific embodiment of the invention, the human hepatocellular carcinoma cell line is a HepG2 cell line. Such HepG2 cell lines are commercially available from the American Tissue Culture Center as accession number HB-8065. In yet another embodiment of the invention, derivatives of HepG2 cells may be used in the methods of the invention. Derivatives include those cell lines that have been genetically modified but which retain the ability to efficiently produce the recombinant polypeptide of interest.

A “cell” usually refers to a single cell, which however can be part of a culture of cells. A “cell culture” generally refers to a plurality of cells being cultured in a culture medium, the cells preferably being derived from a single cell clone. The term “cell line” is used mainly as a general covering term for cells derived from a single cell that can be grown permanently and from which cells and cell cultures can be obtained. A permanent cell is often referred to as an immortalized cell, or as a continuous cell. By contrast, a primary cell means a cell that has been obtained from an organism and possibly subculturing and has only a limited lifetime (usually about 20 cell generations or less). A permanent hepatocellular liver carcinoma cell can for instance be obtained from a primary liver carcinoma sample. Preferably, the cells according to the invention are cells, i.e. derived from primary hepatocellular liver carcinoma tissue.

In a preferred embodiment of the invention, infection wherein a replication-defective recombinant adenovirus is used as a means for introducing the nucleic acid encoding the polypeptide of interest into a cell is used. In a preferred embodiment, the infection of the cell results in transduction allowing for efficient production of the polypeptide of interest.

As used herein, the term “infecting” means exposing the recombinant adenovirus to the cell under conditions so as to facilitate the infection of the cell with the recombinant adenovirus. By way of example, and not limitation, a virus concentration in the range of 10⁶ to about 10¹⁰, preferably in the range of about 10⁸ to about 10⁹ virions per ml can be used. Chemical agents may also be employed to increase the infectivity of the cell line. For example, the present invention provides a method to increase the infectivity of cell lines for viral infectivity by the inclusion of a calpain inhibitor. Examples of calpain inhibitors useful in the practice of the present invention include calpain inhibitor 1 (also known as N-acetyl-leucyl-leucyl-norleucinal, commercially available from Boehringer Mannheim). Calpain inhibitor 1 has been observed to increase the infectivity of cell lines to recombinant adenovirus.

By way of example, and not limitation, if viral vector stock is limiting infection can be done in a minimal volume for 2-4 hours followed by addition of media for the production phase as the majority of infection will occur in the first few hours. Rocking of the cell culture vessels at low speed during the infection can also help increase infection efficiency.

Also by way of example, and not limitation, for cell factories, full media volume, containing the adenovirus vector at a concentration of 5×10⁸ particles/ml, can be added to the cells permitting infection at that optimal vector-density for longer period of time. This can require more vector and result in more vector-derived contaminants in the preparations.

As used herein, the term “culturing under conditions to permit expression of the polypeptide of interest” means maintaining the conditions for the cell line infected with recombinant adenovirus so as to permit the virus to express the polypeptide of interest. It is desirable to control conditions so as to maximize the production of polypeptide produced by each cell. Consequently it will be necessary to monitor and control reaction conditions such as temperature, dissolved oxygen, pH, etc. Commercially available bioreactors such as the CelliGen Plus Bioreactor (commercially available from New Brunswick Scientific, Inc. 44 Talmadge Road, Edison, N.J.) have provisions for monitoring and maintaining such parameters. Optimization of infection, transfection and culture conditions will vary somewhat, however, conditions for efficient polypeptide production may be achieved by those of skill in the art taking into consideration, for example, the known properties of the cell line, properties of the virus and the type of bioreactor.

Cell culture media are available from various vendors, and serum-free culture media are nowadays often used for cell culture, because they are more defined than media containing serum and easier for downstream purification. The cells of the present invention preferably grow well in serum-containing media as well as in serum-free media. The conditions for growing or multiplying cells (see, e.g., Tissue Culture, Academic Press, Kruse and Paterson, editors (1973)) and the conditions for expression of the recombinant product are known to the person skilled in the art. In general, principles, protocols, and practical techniques for maximizing the productivity of mammalian cell cultures can be found in Mammalian Cell Biotechnology: a Practical Approach (M. Butler, ed., IRL Press, 1991).

Examples of standard cell culture media for laboratory flask or low density cell culture being adapted to the needs of particular cell types are for instance: Roswell Park Memorial Institute (RPMI) 1640 medium (Morre, G., The Journal of the American Medical Association, 199, p. 519 f. 1967), L-15 medium (Leibovitz, A. et al., Amer. J. of Hygiene, 78, 1 p. 173 ff, 1963), Dulbecco's modified Eagle's medium (DMEM), Eagle's minimal essential medium (MEM), Ham's F12 medium (Ham, R. et al., Proc. Natl. Acad. Sc. 53, p 288 ff. 1965) or Iscoves' modified DMEM lacking albumin, transferrin and lecithin (Iscoves et al., J. Exp. med. 1, p. 923 ff., 1978). It is known that such culture media can be supplemented with fetal bovine serum (FBS, also called fetal calf serum FCS), the latter providing a natural source of a plethora of hormones and growth factors. The cell culture of mammalian cells is a routine operation well-described in scientific textbooks and manuals, it is covered in detail e.g. in R. Ian Fresney, Culture of Animal cells, a manual, 4th edition, Wiley-Liss/N.Y., 2000. Preferably, the cell culture medium according to the present invention in the production phase, is devoid of fetal calf serum (FCS or FBS), which then is termed “serum-free.” Cells in serum-free medium generally require insulin and transferrin in a serum-free medium for optimal growth.

Hepatocellular liver carcinoma cells, such as HepG2 cells, have been reported to express alpha fetoprotein, albumin, alpha1 anti-trypsin, anti-chymotrypsin, transferring, and plasminogen. The expression of such proteins may facilitate growth in serum free media thereby providing an advantage for production of the polypeptide of interest in such cell lines rather than cell lines from other tissues.

A possible embodiment of one method of the present invention, namely expression and harvest of the polypeptide of interest, is high-density growth of the mammalian host cells e.g. in an industrial fed-batch bioreactor. The host cells may be cultured in any suitable vessel which is known in the art. For example, cells may be grown and the infected cells may be cultured in a biogenerator or a bioreactor. Generally, “biogenerator” or “bioreactor” means a culture tank, generally made of stainless steel or glass, with a volume of 0.5 liter or greater, comprising an agitation system, a device for injecting a stream of CO₂ gas and an oxygenation device. Typically, it is equipped with probes measuring the internal parameters of the biogenerator, such as the pH, the dissolved oxygen, the temperature, the tank pressure or certain physicochemical parameters of the culture (for instance the consumption of glucose or of glutamine or the production of lactate and ammonium ions). The pH, oxygen, and temperature probes are connected to a bioprocessor which permanently regulates these parameters. In other embodiments, the vessel is a spinner flask, a roller bottle, a shaker flask or in a flask with a stir bar providing mechanical agitation. In another embodiment, the vessel is a WAVE Bioreactor (WAVE Biotech, Bridgewater, N.J., U.S.A.). In yet another embodiment fiber bed discs (Fibra-cell) are used in the present invention.

In one embodiment, a high-density growth culture medium can be employed. Such high-density growth media can usually be supplemented with nutrients such as all amino acids, energy sources such as glucose in the range given above, inorganic salts, vitamins, trace elements (defined as inorganic compounds usually present at final concentrations in the micromolar range), buffers, the four nucleosides or their corresponding nucleotides, antioxidants such as Glutathione (reduced), Vitamin C and other components such as important membrane lipids, e.g. cholesterol or phosphatidylcholine or lipid precursors, e.g. choline or inositol. A high-density medium will be enriched in most or all of these compounds, and will, except for the inorganic salts based on which the osmolarity of the essentially isotonic medium is regulated, comprise them in higher amounts (fortified) than the afore mentioned standard media.

In a specific embodiment of the invention, the cells, such as, but not limited to, hepatocellular carcinoma cells may be grown attached to the surface of a substrate. Such substrates include, for example, microcarriers, fiber beds, hollow fiber cartridges and stacked plate modules can be used for attachment dependent cell culture scale up. Such microcarriers include, for example, glass beads with a surface treatment to enhance cell attachment. Microcarriers are used with stirred vessels/tanks, having sufficient agitation to keep the carriers in motion in the media and prevent settling. Alternatively, fiber beds comprising flat discs made of fiber can be used in stirred vessels/tanks where the cells adhere to the fiber surface. In such instances, media is forced through the bed via a stirrer mechanism thereby delivering nutrients to the cells. Hollow fiber cartridges comprising multiple hollow fibers may also be used to culture cells. Cells reside on the outside of the fibers and nutrients flow through the lumen of the hollow fiber and pass through the fiber membrane to the cells. Stacked plate modules comprising plates made of plasma treated styrene sealed into a module may also be used for culturing of the hepatocellular liver carcinoma cells. Cells attach to the plate surface while a unique low shear flow design delivers media evenly across the cells.

Polypeptides may be produced by culturing the cells; optionally adding fresh growth medium to the cells; inoculating the cells with the virus; incubating the inoculated cells; optionally adding fresh growth medium to the inoculated cells; and harvesting the polypeptide of interest from the cells and/or the medium. By way of example, and not limitation, the polypeptide of interest can be harvested between about 2 to 5 days, such as, for example, 4 days.

Since the cells used in the methods herein are not permissive for replication of adenovirus, preferably not permissive for replication defective adenovirus, the infected cells can be grown for prolonged periods with greatly reduced levels of cytotopathology. By way of example, and not limitation, the polynucleotide of interest can be harvested from between about 2 to about 5 times, preferably between about 3 to about 4 times. In a specific embodiment of the invention, the infected hepatocellular liver carcinoma cells may be re-infected multiple times with recombinant adenovirus to increase the production of the polypeptide of interest. By way of example, and not limitation, the cells can be reinfected between about 1 to about 4 times. Supernatant may be collected prior to re-infection for isolation of the polypeptide of interest, followed by the addition of new medium.

Expression of the polypeptide of interest can be determined by various indexes including, but not limited to the use of assays such as, for example, immunostaining immunoprecipitation and immunoblotting, enzyme-linked immunosorbent assay, nucleic acid detection (e.g., Western blot analysis , Southern blot analysis, Northern blot analysis,).

Polypeptides produced by the infected cells may also be isolated and purified. Proteins, polypeptides and peptides produced by recombinant adenoviruses may be purified by standard methods, including, but not limited to, salt or alcohol precipitation, affinity, preparative disc-gel electrophoresis, isoelectric focusing, high pressure liquid chromatography (HPLC), reversed-phase HPLC, gel filtration, cation and anion exchange and partition chromatography, and countercurrent distribution. Such purification methods are well known in the art and are disclosed, e.g., in “Guide to Protein Purification”, Methods in Enzymology, Vol. 182, M. Deutscher, Ed., 1990, Academic Press, New York, N.Y. In one embodiment, supernatants are stored at 4° C. or −20° C. depending on stability and time to purification of the protein of interest. In another embodiment, where the protein of interest is not secreted into the medium, the cells may be harvested, for example through centrifugation, and lysed to release the protein of interest.

The present invention encompasses compositions comprising a hepatocellular carcinoma liver cell line containing a recombinant adenovirus (preferably, replication-defective recombinant adenovirus) wherein said recombinant adenovirus expresses a polypeptide of interest. In a preferred embodiment of the invention, the hepatocellular liver carcinoma cell is a human cell. In a specific embodiment of the invention, the human hepatocellular liver carcinoma cell line is a HepG2 cell line. Such a HepG2 cell line is commercially available from the American Tissue Culture Center as accession number HB-8065. In yet another embodiment of the invention, derivatives of HepG2 cells may be used in the methods of the invention. Derivatives include those cell lines that have been genetically modified but which retain the ability to efficiently produce the recombinant polypeptide of interest.

The invention further relates to a culture of hepatocellular carcinoma cells infected with a recombinant adenovirus genetically engineered to express a protein, polypeptide or peptide of interest. Such compositions can be used in vitro to efficiently express proteins, polypeptides and peptides of interest.

Pharmaceutical Compositions

The invention further comprises pharmaceutical compositions comprising a polypeptide of interest, produced by the methods of the invention, and a pharmaceutically acceptable carrier. Generally, a polypeptide with desired therapeutic and/or prophylactic and/or diagnostic purposes may be a preferred. In a specific embodiment, the term “pharmaceutically acceptable carrier” means approved by a regulatory agency of the Federal or a state government or listed in the U.S. Pharmacopeia or other generally recognized pharmacopeiae for use in animals, and more particularly in humans. The term “carrier” refers to a diluent, adjuvant, excipient, or vehicle with which the pharmaceutical composition is administered. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. These compositions can take the form of solutions, suspensions, emulsion, tablets, pills, capsules, powders, sustained-release formulations and the like. These compositions can be formulated as a suppository. Oral formulation can include standard carriers such as pharmaceutical recombinant adenoviruses of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain an effective amount of recombinant adenovirus, preferably in purified form, together with a suitable amount of carrier so as to provide the form for proper administration to the patient. The formulation should suit the mode of administration.

The amount of the pharmaceutical composition of the invention which will be effective in the treatment of a particular disorder or condition will depend on the nature of the disorder or condition, and can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. Effective doses may be extrapolated from dose-response curves derived from in vitro or animal model test systems.

Methods of administration of the compositions include, but are not limited to, intradermal, intramuscular, intraperitoneal, intravenous, subcutaneous, intranasal, epidural, and oral routes. The pharmaceutical compositions of the present invention may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compositions of the invention into the lungs by any suitable route. Pulmonary administration can be employed, e.g., by use of an inhaler or nebulizer, and formulation with an aerosolizing agent.

In a specific embodiment, it may be desirable to administer the pharmaceutical compositions of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. In one embodiment, administration can be by direct injection at the site (or former site) of a malignant tumor or neoplastic or pre-neoplastic tissue.

In another embodiment, the pharmaceutical composition can be delivered in a controlled release system. In one embodiment, a pump may be used (see Langer, supra; Sefton, 1987, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger & Peppas, 1983, J. Macromol. Sci. Rev. Macromol. Chem. 23:61; see also Levy et al., 1985, Science 228:190; During et al., 1989, Ann. Neurol. 25:351(1989); Howard et al., 1989, J. Neurosurg. 71:105). In yet another embodiment, a controlled release system can be placed in proximity of the composition's target, i.e., the lung, thus requiring only a fraction of the systemic dose (see, e.g., Goodson, 1984, in Medical Applications of Controlled Release, supra, vol. 2, pp. 115-138). Other controlled release systems are discussed in the review by Langer (1990, Science 249:1527-1533).

### EXAMPLE

### Efficient Adenovirus Mediated Expression of Interferon in HEPG2 Cells

This method provides a simple and rapid method for production of a polypetide utilizing non-permissive cells to maximize polypeptide protein production output. Prior reports have utilized 293-based cells and other cells that encode genes capable of supporting rAd gene expression and replication through transcomplementary transcription regulatory function. For example the presence of a stable gene encoding the rAd E1a gene in 293 cells enables replication defective rAd-viruses to replicate. The caveat to the use of replication permissive cells is that this promotes cytopathic effects (CPE) following infection, thus, limiting overall protein production due to the induction of apoptosis.

To improve the process a number of non-permissive cell lines, incapable of promoting recombinant adenovirus DNA and viral replication were tested for their ability to express a polypeptide of interest. This strategy enables production over a longer period of time and permits multiple harvests of protein from supernatants following transduction. Moreover, less contaminating recombinant adenovirus-derived proteins may be translated thereby facilitating purification of the specific protein of interest. To improve the process we screened non-permissive cell lines, incapable of promoting rAd DNA and viral replication. As described below, of the cell lines tested, HepG2 cells ( ATCC # HB-8065) were readily infected and produced the highest concentration of secreted cytokine (hybrid interferon, IFN A titration of optimal vector concentration revealed optimal transduction of HepG2 cells at 5×10⁸ particles/ml. High transduction efficiency and minimal cell damage can be achieved at this concentration.

### Materials and Methods

HepG2 cells from ATCC were grown in Eagle's Minimum Essential Medium supplemented with glutamine (2 mM), sodium pyruvate (1 mM), nonessential amino acids (0.1 mM) and 10% FCS (certified low immunoglobulin content). HepG2 cells also were grown in DME high glucose supplemented with glutamine and 10% FCS. Following initial expansion of cells in the medium described above, protein production was done in DME high glucose (4.5 g/l)supplemented with glutamine (2 mM), and CMF serum free media.

In a representative experiment, HepG2 cells were expanded in flat stock (T-165s or T-225 flasks), triple flasks, or cell factories (5× triple flasks per cell factory). Cells were allowed to expand to a density approaching 85-90% of confluency. The high density is preferred as the recombinant adenovirus will not replicate in the HepG2 cells and thus new progeny cells will not be infected with nacent vector and DNA will be rapidly diluted out upon cell division. Cells (at about 85% confluency) were infected with recombinant adenovirus encoding the protein of interest in medium diluted to a concentration of 5×10⁸ particles/ml. The infection was carried out with just enough volume to cover the cells at 37° C. for 2-4 hours (i.e., 10 mls in a T-165 flask). Care was taken to make sure the flask is level to avoid “dry spots”. Media was then added to the cells for incubation for 4-5 days during which protein production ensued. At the end of this period, the supernatant was collected and the protein isolated from the media. Fresh medium was added to the transduced cells for an additional round of protein production and harvest at this time point. Supernatants were again harvested following 4-5 days of additional growth. The supernatants were centrifuged (2000-3000 RPM) for 10 minutes to remove cells and debris and then filtered using 0.2 or 0.45 micron filtration systems.

Three replication non-permissive cell lines, 4T1, HepB3 and HepG2, were tested for production of hybrid interferon after their inoculation with a recombinant replication deficient adenovirus carrying a hybrid interferon expression cassette (IHCB; Cancer Gene Ther:2006 Jul;13(7):664-75. Epub 2006 Mar 3; J Interferon Cytokine Res. 2001 Jun;21(6):399-408). Protein production was performed in serum free media to simplify protein purification. Of the three lines tested, the HEpG2 cells survived the longest and also produced the most hybrid interferon.

In a representative experiment, when HepG2 cells were grown in flat stock culture conditions, the cells produced approximately 10 fold more interferon protein than 293 cells grown under the same conditions or roughly the same as 293 cells grown in a bioreactor where the cell density was 10 fold higher. Since the HepG2 cells are not permissive to adenovirus replication, and the culture continued to grow after infection the interferon containing supernatant was removed after 3 to 4 days, and the cells re-fed and infected a second time, for a second harvest equal to the first. This was repeated for a third harvest. Protein production was assessed by ELISA. After each of the first two infections approximately 16-20 mg of protein was produced per liter of culture volume. After the third infection the yield was reduced because the cells lifted from the plastic cell factories.

In a representative experiment, production of proteins other than hybrid IFN (i.e., universal IFN, IFNA/D or IFNα2α1) was also tested using HepG2 cells and replication deficient adenovirus. In particular, both IFNα2b and GMC-SF were successfully expressed and secreted. After single infection, IFNα2b containing supernatant was collected and cells were re-fed every two days. Sustained protein production was observed for over a week and on day ten post infection it started to decline due to limited adenoviral gene expression.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

Patents, patent applications, publications, product descriptions, Genbank Accession Numbers and protocols are cited throughout this application, the disclosures of which are incorporated herein by reference in their entireties for all purposes.

1. A method for production of a polypeptide, said method comprising culturing a replication non-permissive mammalian cell containing a recombinant adenovirus comprising a nucleotide sequence encoding a polypeptide of interest under conditions so as to permit expression of the polypeptide in said cell.

2. The method of claim 1, wherein the cell is a hepatocellular carcinoma cell.

3. The method of claim 2, wherein the cell is a HepG2 cell is ATCC No.:8065).

4. The method of claim 1, wherein the recombinant adenovirus is a replication defective adenovirus.

5. The method of claim 4, wherein the recombinant adenovirus comprises a protein IX deletion.

6. The method of claim 1, further comprising the steps of harvesting the cells.

7. The method of claim 1, further comprising the step of recovering the polypetide.

8. The method of claim 1, wherein the polypeptide is a cytokine.

9. The method of claim 1, wherein the polypetide is an antibody.

10. A pharmaceutical composition comprising the polypeptide of claim 7, 8 or 9.
